(19) United States
(12) Patent Application Publication
(10) Pub. No.: US 2010/0221767 A1
(43) Pub. Date: Sep. 2, 2010

(54) DIAGNOSTIC METHOD AND PROGNOSTIC TOOL FOR OSTEOARTHRITIS

(76) Inventors: Morris Frank MANOLSON, Toronto (CA); Svetlana Komarova, Kirkland (CA); Rene Harrison, Toronto (CA); Artur De Brum Fernandes, Sherbrooke (CA); Gilles Boire, Sherbrooke (CA); Lukasz Kurgan, Edmonton (CA); S. Jeffrey Dixon, London (CA); Stephen M. Sims, London (CA); Diana P. Trebec-Reynolds, Toronto (CA)

Correspondence Address: McCarthy Tetrault LLP, Box 48, Suite #4700 Toronto Dominion Bank Tower, TORONTO, ON, M5K 1E6, CA

(21) Appl. No.: 12/581,052

(22) Filed: Oct. 16, 2009

Related U.S. Application Data
(60) Provisional application No. 61/106,001, filed on Oct. 16, 2008.

(51) Int. Cl. C12Q 1/02

(52) U.S. Cl. 435/29

(57) ABSTRACT

The invention relates to a diagnostic method and prognostic tool for osteoarthritis and uses thereof.

### RELATED APPLICATIONS

This application claims the benefit of priority of U.S. provisional application No. 61/106,001, filed Oct. 16, 2008, entitled “Diagnostic Method and Prognostic Tool For Osteoarthritis” which is incorporated by reference herein.

### FIELD OF THE INVENTION

The invention relates to a diagnostic method and prognostic tool for osteoarthritis and uses thereof.

### BACKGROUND OF THE INVENTION

Bone is a central element in the pathophysiology of degenerative and inflammatory arthropathies. Bones play an important role in the pathophysiology of osteoarthritis (OA). Subchondral bone sclerosis, osteophytes and bone cysts are hallmarks of OA. Subchondral bone remodeling activity correlates with joint space narrowing (Dieppe, P., J. Cushnaghan, P. Young and J. Kirwan. 1993. Prediction of the progression of joint space narrowing in osteoarthritis of the knee by bone scintigraphy. Ann Rheum Dis 52:557-563) and may precede cartilage changes in a spontaneous animal model of OA (Carlson, C. S., R. F. Loeser, M. J. Jayo, D. S. Weaver, M. R. Adams and C. P. Jerome. 1994. Osteoarthritis in cynomolgus macaques: a primate model of naturally occurring disease. J Orthop Res 12:331-339). Some authors have recently hypothesized that changes in bone metabolism and mechanical properties could contribute to the genesis of OA. Interestingly, subchondral osteoblasts from knees with OA present an altered phenotype (Hilal, G., J. Martel-Pelletier, J. P. Pelletier, P. Ranger and D. Lajeunesse. 1998. Osteoblast-like cells from human subchondral osteoarthritic bone demonstrate an altered phenotype in vitro. Arthritis Rheum 41:891-899) that can also be observed in osteoblasts obtained from the iliac crest of OA patients (Dequeker, J., S. Mohan, R. D. Finkelman, J. Aerssens and D. J. Baylink. 1993). Generalized osteoarthritis is associated with increased insulin-like growth factor types I and II and transforming growth factor beta in cortical bone from the iliac crest (Dequeker, J., S. Mohan, R. D. Finkelman, J. Aerssens and D. J. Baylink. 1993. Generalized osteoarthritis associated with increased insulin-like growth factor types I and II and transforming growth factor beta in cortical bone from the iliac crest. Possible mechanism of increased bone density and protection against osteoporosis. Arthritis Rheum 36:1702-1708.), suggesting that a systemic dysfunction of osteoblasts could be implicated in the pathogenesis of OA. Other phenotypic and metabolic changes have also been described in osteoblasts from OA joints (Lavigne, P., M. Benderdour, D. Lajeunesse, Q. Shi and J. C. Fernandes. 2004. Expression of ICAM-1 by osteoblasts in healthy individuals and in patients suffering from osteoarthritis and osteoporosis. Bone 35:463-470; Massicotte, F., J. C. Fernandes, J. Martel-Pelletier, J. P. Pelletier and D. Lajeunesse. 2006. Modulation of insulin-like growth factor 1 levels in human osteoarthritic subchondral bone osteoblasts. Bone 38:333-341). Although the primary role of subchondral bone in the pathogenesis of OA remains a matter of debate, it is clear that intense bone remodeling occurs both in human OA, as shown early in the course of disease in humans by technetium-99m scans (McCrae, F., J. Shouls, P. Dieppe and I. Watt. 1992. Scintigraphic assessment of osteoarthritis of the knee joint. Ann Rheum Dis 51:938-942), and in animal models of the disease (Lavigne, P., M. Benderdour, D. Lajeunesse, P. Reboul, Q. Shi, J. P. Pelletier, J. Martel-Pelletier and J. C. Fernandes. 2005. Subchondral and trabecular bone metabolism regulation in canine experimental knee osteoarthritis. Osteoarthritis Cartilage 13:310-317).

OA is the leading cause of physical disability, decreased quality of life and increase in health care utilization in industrialized societies (Herndon J H, Davidson S M, Apazidis A. Recent socioeconomic trends in orthopaedic practice. J Bone Joint Surg Am 83-A(7):1097-1105, 2001). Knee OA alone leads to disability as often as heart disease and chronic obstructive lung disease (Guccionne A A, Felson D T, Anderson J J et al. The effects of specific medical conditions on the functional limitations of elders in the Framingham Study. Am J Public Health 84:351-357, 1994). Radiologic OA can be found in up to 90% of all person aged 40 and older, and is estimated that 8% of the population currently suffer from symptomatic OA (Moskowitz R W, Holderbaum D. Clinical and Laboratorial Findings in Osteoarthritis. In Koopman W J: Arthritis and Allied Conditions. Lippincott Williams & Wilkins, Philadelphia Pa., 2001. p 2217) (about 56 million people). The degree of severity and progression varies from radiologic signs and absence of symptoms to important pain and functional limitation. The diagnosis of OA relies on the clinical evaluation and radiologic findings. There are no tests to assess the prognosis of OA.

Current treatment of OA includes reducing the pain and increasing function with physiotherapy and, eventually, surgery for joint replacement. However, no treatment presently exists capable of reducing the rate or of stopping the progression of the disease.

As such, there is a need for a diagnostic method for OA and the ability to determine and/or predict the severity of OA. The development of a prognostic tool to allow for prediction of progression of disease and a method that would enable the monitoring of same over time or throughout a treatment regime in a patient would also be useful. This would assist in the clinical management and/or treatment of patients and to tailor treatment regimes for them.

### SUMMARY OF THE INVENTION

In one embodiment of the invention, it has been found that, in individuals with OA, osteoclasts differentiated from the PBMC present lower apoptosis rate than a control group composed of self-reported healthy volunteers. In another embodiment, it was found that individuals with OA, osteoclasts differentiated from the PBMC present more bone resorption than a control group composed of self-reported healthy volunteers. In yet another embodiment it was found that OC expressed proteins can be used as indicators of OA.

The present invention relates to a method for the diagnosis and/or prognosis of osteoarthritis (OA) in a subject comprising assaying a sample from the subject and measuring: (1) apoptosis of osteoclasts generated from peripheral blood mononuclear cells from said sample, and/or (2) resorptive activity of said osteoclasts, and/or (3) OC expressed proteins such as IL-1R1, IL-1R2; IL-1R1/IL-1R2; RANK wherein a change in any such values as compared to values from a control sample is useful in the diagnosis and/or prognosis of OA and/or disease state, including functional status. In one embodiment a decrease in osteoclast apoptosis or OC expressed proteins and/or an increase in osteoclast resorptive activity compared to a control sample, in one embodiment a control sample of normal or non-OA subjects, is indicative of OA. In one embodiment the values predict the presence and/or severity of OA. Surrogate markers of osteoclast apoptosis or osteoclast resorption activity can be used for the same purposes.

In one embodiment, one or more of the following markers and/or OC expressed proteins can be used as a marker of OA: actual cell apoptosis; IL-1R1, IL-1R2; IL-1R1/IL-1R2; RANK. For the markers IL-1R1, IL-1R2 and RANK, they are expressed herein as a ratio to GAPDH, e.g. IL-1R1/GAPDH, IL-1R2/GAPDH; RANK/GAPDH. GAPDH is used as a control as it is a ubiquitously expressed metabolic protein. By expressing the markers as a ratio to GAPDH, it is insured that variations in expression level are not due to artifacts. In one embodiment, more than one marker can be used (e.g. plotted on an X/Y graph) to enhance diagnosis and/or prognosis of OA, such as shown, but not necessarily limited to the combinations shown in Example 6 herein.

The invention also relates to a method of screening a subject for OA comprising: (a) obtaining a biological sample from a subject, in one embodiment a sample comprising osteoclast precursors, such as human peripheral blood mononuclear cells; (b) detecting the amount of osteoclast apoptosis and/or osteoclast resorptive activity and/or OC expressed proteins such as IL-1R1, IL-1R2; IL-1R1/IL-1R2; RANK in said sample; and (c) comparing said values of osteoclast apoptosis and/or osteoclast resorptive activity detected and/or OC expressed proteins to a predetermined normal, where detection of a decrease in osteoclast apoptosis and/or an increase in osteoclast resorptive activity and/or OC expressed proteins compared to a control sample is indicative of the presence, prognosis, and/or severity of OA.

In one embodiment, the “sample” or “biological sample”, is any material known to or suspected of expressing or containing osteoclasts, its precursors or surrogate markers of the presence, differentiation capacity, activity or apoptosis of these cells. The test sample can be used directly as obtained from the source or following a pretreatment to modify the character of the sample. In one embodiment, the biological sample is a blood sample or serum or tissue extracts. In one embodiment it is serum or a fraction thereof, or any sample comprising an osteoclast precursor, such as peripheral blood mononuclear cells (PBMCs).

The term “subject” refers to a warm-blooded animal such as a mammal which is afflicted with or suspected to be afflicted with osteoarthritis. Preferably, “subject” refers to a human.

The invention also relates to kits for carrying out the methods of the invention.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1A is a bar graph illustrating the number of osteoclasts per well in control versus OA subjects as described in Example 1.

FIG. 1B is a bar graph illustrating the number of osteoclasts/well divided by the number of CD 14+ cells (osteoclast precursors) in the blood sample studied in control versus OA subjects as described in Example 1.

FIG. 2 is a bar graph illustrating the percentage of osteoclasts in apoptosis after the cells were kept for 24 h without M-CSF and RANKL in 5% FBS in controls (non-OA subjects) versus OA subjects as described in Example 2.

FIG. 3 is a bar graph illustrating the resorption activity of osteoclasts in control (non-OA subjects) versus OA subjects as described in Example 3.

FIG. 4 is a bar graph illustrating data with youngest from control group removed for: RANK/GAPDH (A); IL-1R1/GAPDH (B); IL-1R2/GAPDH (C); IL-1R1/IL-1R2 (D); and Apoptosis (E)

FIG. 5 is a bar graph illustrating data with oldest OA patients removed for: RANK/GAPDH (A); IL-1R1/GAPDH (B); IL-1R2/GAPDH (C); IL-1R1/IL-1R2 (D); and Apoptosis (E)

FIG. 6 is a bar graph illustrating data for all control and OA patients (no patients or controls removed) for: RANK/GAPDH (A); IL-1R1/GAPDH (B); IL-1R2/GAPDH (C); IL-1R1/IL-1R2 (D); and Apoptosis (E)

FIG. 7 is a graph illustrating the distribution of patients considering RANK/GAPDH and Apoptose values using OA presence rem ctrls (Age matched control where youngest controls were removed) dataset. Only patients who have both Apoptose and RANK/GAPDH values (around 45% of the dataset) were considered. “X” indicate OA patients and indicate controls.

FIG. 8 is a graph illustrating the distribution of patients considering IL-1R1/IL-1R2 and Apoptose values using OA presence rem OAs(Age matched controls where oldest OA patients were removed) dataset. Only patients who have both Apoptose and IL-1R1/IL-1R2 values (around 45% of the dataset) were considered. “X” indicate OA patients and “°” indicate controls.

FIG. 9 is a graph illustrating the distribution of patients considering RANK/GAPDH and Apoptose values using OA presence using the dataset where no controls nor OA patients were removed. Only patients who have both Apoptose and RANK/GAPDH values (around 45% of the dataset) were considered. “X” indicate OA patients and “°” indicate controls.

### DETAILED DESCRIPTION OF THE INVENTION

Osteoarthritis (OA) is associated with periarticular bone loss and erosions which contribute to joint destruction to which osteoclasts (OC) are critical. Although the involvement of OC in bone and joint destruction in OA has been confirmed, their role in disease onset and progression was previously not known.

Osteoclasts are normally involved in bone remodelling, through a process of matrix attachment, and bone resorption based on an acid secretion mechanism that has been previously described (Teitelbaum, S. L., Y. Abu-Amer and F. P. Ross. 1995. Molecular mechanisms of bone resorption. J Cell Biochem 59:1-10; Stenbeck, G. 2002. Formation and function of the ruffled border in osteoclasts. Semin Cell Dev Biol 13:285-292; Blair, H. C. 1998. How the osteoclast degrades bone. Bioessays 20:837-846). These cycles continue until programmed death (apoptosis) of OCs occur. The longer the OCs live the more bone resorption. In fact, delayed apoptosis is one of the main causes of post-menopausal Osteoporosis, where the decreased levels of estrogens, hormones that usually increase OC apoptosis, allow these cells to live longer and to resorb more bone (Krum, S. A., Brown, M. 2008. Unraveling estrogen action in osteoporosis. Cell Cycle 7:1348-1352).

Subchondral bone is an integral part of joints, and it has been hypothesized by some authors that changes in its characteristics might be part of the pathophysiology of Osteoarthritis. In fact, increased bone remodeling is an early finding using scintigraphy in early OA (Vanharanta, H., Kuusela, T., Kiuru, A. 1984. Early detection of developing Osteoarthritis by scintigraphy: an experimental study on rabbits. Eur J Nucl Med 9:426-428.). The present inventors herein show that OCs differentiated from peripheral blood mononuclear cells from patients with OA present lower levels of apoptosis than an age-matched population. This finding is important to better understand the pathophysiology of Osteoarthritis and can be used, using in vitro osteoclastogenesis or simpler surrogate markers of this parameter, for the diagnosis and prognosis of this disease. Surrogate markers of the disease can include those shown in Examples 5 and 6 herein.

Bone health depends on a tight equilibrium between bone formation by osteoblasts and bone resorption by OCs. OC precursors migrate to a resorption site, where they differentiate and fuse, forming multinucleated OCs. OC attachment to bone matrix, mediated by membrane-bound integrins, forms a tightly sealed extracellular compartment where secretion of acid dissolves the bone mineral, and secretion of proteolytic enzymes digests the organic matrix. OCs may be induced to differentiate from OA synovial membrane (Danks, L., A. Sabokbar, R. Gundle and N. A. Athanasou. 2002. Synovial macrophage-osteoclast differentiation in inflammatory arthritis. Ann Rheum Dis 61:916-921; Suzuki, Y., Y. Tsutsumi, M. Nakagawa, H. Suzuki, K. Matsushita, M. Beppu, H. Aoki, Y. Ichikawa, et al. 2001. Osteoclast-like cells in an in vitro model of bone destruction by rheumatoid synovium. Rheumatology (Oxford) 40:673-682) and bone resorption is increased in women with progressive knee OA compared to non-progressive disease (Bettica, P., G. Cline, D. J. Hart, J. Meyer and T. D. Spector. 2002. Evidence for increased bone resorption in patients with progressive knee osteoarthritis: longitudinal results from the Chingford study. Arthritis Rheum 46:3178-3184). A role for OCs in the pathophysiology of OA is further supported by the chondroprotective effect of alendronate in an experimental model of OA (Hayami, T., M. Pickarski, G. A. Wesolowski, J. McLane, A. Bone, J. Destefano, G. A. Rodan and T. Duong le. 2004. The role of subchondral bone remodeling in osteoarthritis: reduction of cartilage degeneration and prevention of osteophyte formation by alendronate in the rat anterior cruciate ligament transection model. Arthritis Rheum 50:1193-1206) and by an association between alendronate intake, decreased knee pain and less subchondral bone abnormalities in women with OA (Carbone, L. D., M. C. Nevitt, K. Wildy, K. D. Barrow, F. Harris, D. Felson, C. Peterfy, M. Visser, et al. 2004. The relationship of antiresorptive drug use to structural findings and symptoms of knee osteoarthritis. Arthritis Rheum 50:3516-3525). However, the role of OCs in the pathogenesis of OA was previously unknown.

The present inventors have now surprisingly shown that the capacity to generate OC from PBMC in culture, the degree of OC apoptosis, OC expressed protein levels, and resorption activity can independently or in combination be used as diagnostic and/or prognostic indicators of OA. In one embodiment, the inventors have shown that OC values derived from culturing Human peripheral blood mononuclear cells (PBMCs) taken from blood samples from patients and their subsequent degree of apoptosis of said OCs, OC expressed protein levels and/or resorption activity can be used as diagnostic indicators of OA and severity and activity of same.

Human peripheral blood mononuclear cells (PBMCs) can differentiate into OC capable of bone resorption. The capacity for in vitro osteoclastogenesis varies widely in a normal human population, distinguishing two subgroups, high and low differentiators, regardless of age, gender, weight, or other demographic variables.

In one embodiment, the present inventors have herein shown that a decrease in the apoptosis rate and/or decrease in OC expressed proteins, such as IL-1R1, IL-1R2; IL-1R1/IL-1R2; RANK, and/or an increase in bone resorption capacity by OC generated in vitro from peripheral blood mononuclear cells correlates with the diagnosis of OA and that this parameter or any other parameter derived from it and being able to predict the osteoclast characteristics of apoptosis rate and bone resorption capacity of an individual could be used for the early diagnosis and prognosis of OA. Such information is useful in the development of a treatment regime for a subject in need thereof and/or to optimize a treatment regime in a subject, in one embodiment, for monitoring the effectiveness of same by monitoring changes in OC apoptosis and/or OC expressed proteins as disclosed herein and/or resorption in samples of a subject over time or as compared to internal and/or external controls. Said controls including but not limited to subjects or a population of subjects with OA on different treatment regimes or no treatment and/or subjects without OA who are subject to the same or no treatment regime. For instance, wherein an increase in or higher value of OC resorption and/or a decrease in apoptosis or OC expressed proteins (such as those disclosed in the Examples herein) as compared to a control (e.g. prior values of said subject or others with OA on no or other treatment regimes or to values of a normal subject or population (without OA)), can be indicative of less effective treatment for said subject as compared to a control, whereas the lower values in a treatment population versus a non-treatment or other treatment population (with OA or without OA) or lowering of values of OC resorption and/or an increase in apoptosis or OC expressed proteins (such as those disclosed in the Examples) as compared to said controls can be indicative of a more effective treatment regime and/or amelioration of a disease state in said subject.

OA, herein, was diagnosed according to the ACR Criteria for knee OA (Altman R, Asch E, Bloch D, Bole G, Borenstein D, Brandt K, Christy W, Cooke T D, Greenwald R, Hochberg M, et al. Development of criteria for the classification and reporting of osteoarthritis. Classification of osteoarthritis of the knee. Diagnostic and Therapeutic Criteria Committee of the American Rheumatism Association. Arthritis Rheum. 1986 Aug 29(8):1039-1049.). The patients in this study were recruited at the Division of Rheumatology, and all signed an informed consent. The control group was constituted of self-reported healthy individuals from the local (Sherbrooke) who volunteered to participate and signed informed consents. All clinical and laboratorial data are kept in a secure electronic database in Sherbrooke. Functional status was defined in accordance with WOMAC (Bellamy, N., W. W. Buchanan, C. H. Goldsmith, J. Campbell and L. W. Stitt. 1988. Validation study of WOMAC: a health status instrument for measuring clinically important patient relevant outcomes to antirheumatic drug therapy in patients with osteoarthritis of the hip or knee. J Rheumatol 15:1833-1840). Severity of disease was evaluated radiologically according to the Kellgren and Lawrence method for knee OA (Kellgren, J. H. and J. S. Lawrence. 1957. Radiological assessment of osteo-arthrosis. Ann Rheum Dis 16:494-502).

In one embodiment, the invention provides a method for diagnosing OA in a subject and/or determining prognosis of OA in a subject, including, in one embodiment but not necessarily limited to determining the severity of OA in a subject comprising:

(a) obtaining a sample from the subject. In one embodiment the sample is a sample comprising OC precursors, such as PBMCs capable of differentiating to OCs. In one embodiment the sample is a blood and/or serum sample. In one embodiment a minimum of 50 mls is obtained. In one embodiment, the OC precursors, in one embodiment PBMCs, are isolated from the sample to the extent necessary to enable the differentiation of the OC precursors, such as PBMCs to OC. In one embodiment PBMCs are isolated from 50 ml of blood by Ficoll-Hypaque gradient. In one embodiment, the isolated OC precursors, such as PBMCs are cultured under conditions that promote differentiation into OCs, such as described in Example 1, however persons of skill in the art would be familiar with other conditions. In one embodiment, the whole population of PBMCs is plated in 48-well tissue culture plates containing a bone slice or a glass slide, and the cells are allowed to differentiate for 21 days in the presence of recombinant RANKL (75 ng/ml) and M-CSF (10 ng/ml). However the length of culturing (e.g. time, days) or other culturing conditions can vary depending on the amount of OCs desired. Controls should be cultured under the same conditions or values standardized taking into account any variations of culturing conditions; and

(b) measuring and/or determining one or more of the following, wherein in one embodiment at least one of (ii) and (iii) below are measured and/or determined:

(i) amount of OCs derived from the sample, OC precursors or PBMC. This can be determined as described in the Examples although other methods known in the art could be used. In one embodiment, after 21 days in culture the cells are fixed and stained for TRAP activity and with hematoxylin. The number of TRAP+ cells containing three or more nuclei per well in a 48 wells plate is counted in each well. (Durand M, Gallant M A, de Brum-Fernandes A J. Prostaglandin D2 receptors control osteoclastogenesis and the activity of human osteoclasts. J Bone Miner Res. 2008 July; 23(7):1097-10) and are indicative of the amount of OCs derived from PBMCs;

(ii) level of OC apoptosis. Again, various methods for determining same may be known in the art. In one embodiment level of apoptosis can be determined as described in Example 2. In one embodiment, OCs differentiated with M-CSF and RANKL for 21 days are then kept for 24 h without M-CSF and RANKL in 5% FBS. Cell death is visualized using the TACS Blue kit from R&D Systems.

(iii) level of OC expressed proteins, such as those described in Examples 5 and 6. For instance, IL-1R1, IL-1R2; IL-1R1/IL-1R2; RANK. GAPDH can be used as a control, as it is ubiquitously expressed metabolic protein, e.g. IL-1R1/GAPDH, IL-1R2/GAPDH; RANK/GAPDH.

Interleukin-1 (IL-1) is a predominant cytokine in inflammatory conditions such as rheumatoid arthritis and is also involved in osteoclast activation (Dayer J M. 2003: The pivotal role of interleukin-1 in the clinical manifestations of rheumatoid arthritis. Rheumatology (Oxford) 42 Suppl 2:ii3-10. Jimi E, Nakamura I, Duong L T, Ikebe T, Takahashi N, Rodan G A, Suda T. 1999. Interleukin 1 induces multinucleation and bone-resorbing activity of osteoclasts in the absence of osteoblasts/stromal cells. Exp Cell Res 247:84-93. Pacifici R, Carano A, Santoro S A, Rifas L, Jeffrey J J, Malone J D, McCracken R, Avioli L V. 1991. Bone matrix constituents stimulate interleukin-1 release from human blood mononuclear cells. J Clin Invest 87:221-8.). IL-1 has been shown to influence various signaling pathways within osteoclasts, including pro-survival pathways (Jimi E., Shuto T., and Koga T. (1995). Macrophage colony-stimulating factor and interleukin-1 alpha maintain the survival of osteoclast-like cells. Endocrinology 136: 808-811. IL-1 is known to have two receptors, IL-1 receptor type I (IL-1R1) and IL-1 receptor type II (IL-1R2), of which IL-1R1 is the signalling (or activating) receptor and IL-1R2 is a decoy receptor unable to signal due to its lack of a cytoplasmic tail (Dinarello C A. 1998. Interleukin-1, interleukin-1 receptors and interleukin-1 receptor antagonist. Int Rev Immunol 16:457-99.). We have previously shown that both IL-1R1 and IL-1R2 are expressed in the plasma membrane of osteoclasts (Trebec, D., Keying, L., Chandra, D., Gramoun, A., Heersche, J. N. M. and Manolson, M. F. Increased expression of activating factors in large osteoclasts could explain their excessive activity in osteolytic diseases. 2007. J Cell Biochem. 101:205-20).

Here it is shown for the first time that expression levels of IL-1R1 and IL-1R2 are significantly different between a control (healthy) population and patients diagnosed with osteoarthritis. Based on these observations, expression levels of IL-1R1 and IL-1R2 are shown to be useful for the diagnosis or prognosis of osteoarthritis.

RANK (Receptor Activator for Nuclear Factor κ B) is a receptor that is expressed both on pre-osteoclasts and osteoclasts that binds specifically to RANKL. RANKL (Receptor Activator for Nuclear Factor κ B Ligand), (aka: TNF-related activation-induced cytokine (TRANCE), osteoprotegerin ligand (OPGL), and ODF (osteoclast differentiation factor)), is expressed by osteoblasts and immune cells and is the key cytokine involved in osteoclastogenesis. When RANKL binds to RANK on pre-osteoclasts, this turns on the signaling pathway for fusion of the pre-osteoclasts to form multinucleated osteoclasts capable of resorbing bone. When RANKL binds to RANK on mature osteoclasts, this turns on the signaling pathway to begin bone resorption and to prevent apoptosis of osteoclasts. When RANKL no longer binds to RANK on mature osteoclasts, the signaling pathway for apoptosis is turned on and the cells undergo cell death. Here it is shown for the first time that expression levels of RANK are significantly different between a control (healthy) population and patients diagnosed with osteoarthritis. Based on these observations, expression levels of RANK can be useful for the diagnosis and/or prognosis of osteoarthritis;

and/or

(iv) level of OC resorption activity. Various methods for determining same may be known in the art. In one embodiment level of resorption activity can be determined as described in Example 3. In one embodiment, for bone resorption assays, cells differentiated for 21 days on bone slices are stained for TRAP and 0.2% toluidine blue. Resorption surface area is quantified using the image analysis program, Simple PCI. (Durand M, Gallant M A, de Brum-Fernandes A J. Prostaglandin D2 receptors control osteoclastogenesis and the activity of human osteoclasts. J Bone Miner Res. 2008 July; 23 (7): 1097-10); and

(c) determining whether the subject has OA, the prognosis of OA, the severity of OA and/or disease functional status, wherein any changes in OC apoptosis or resorptive activity as compared to a control can be useful in the diagnosis, including prognosis, severity and/or functional status of OA.

In one embodiment:

(i) the level of OC apoptosis is lower than a control; and/or

(ii) the level of OC resorption is higher than a control; and/or

(iii) the level of OC expressed proteins such as IL-1R1; IL-1R2; IL-1R1/IL-1R2; and RANK where GADPH can be used as a control (e.g. IL-1R1/GAPDH; IL-1R2/GADPH; and RANK/GADPH,

is indicative of the subject having OA, and/or is indicative of a more severe and/or aggressive disease state than the control. In one embodiment the control can be a control as described herein. In one embodiment, the control are one or more values obtained from a healthy population of subjects or in one embodiment the mean or normal range of values obtained from said control population.

In one embodiment, a control sample may correspond to OC apoptosis, and/or OC expressed proteins as described herein; and/or osteoclast resorptive activity quantified for samples from healthy, non-OA control subjects, from a random sample of the general population, from samples of subjects with other known OA states (e.g. severe, non-severe OA) or mean or averaged values of all subjects in said selected population with OA or from internal controls using other assayed samples of the subject. In one embodiment the methods of the present invention enable the generation of OC, apoptosis, OC expressed proteins as described herein and OC resorptive values in a number of OA disease states that can establish value ranges for said states (such as the presence, prognosis, severity and/or functional status of OA) that are standardized taken into account the methods in which the values are obtained (e.g. methods for culturing PBMCs under differentiating conditions), that can be used as comparators to determine OA, OA functional status, severity and/or prognosis, by comparing the values obtained from the subject with values obtained previously or simultaneously for others with known disease states, wherein similar values will be indicative of a similar disease state for said subject. It is submitted that a person of skill in the art would appreciate what type of control could be used depending on the purpose of conducting the methods of this invention, whether, for example, it be for initial or subsequent diagnosis of disease state in a person or for monitoring the disease state of a subject over time. For instance, if one wishes to monitor the progression or effectiveness of a treatment in a subject with OA, results from prior samples of the subject could be used as comparators, where a change in values can be indicative of a change of disease state (presence, prognosis, severity and/or functional status). In one embodiment, an increase in OC, resorption and/or a decrease in apoptosis or OC expressed proteins as described herein could be indicative of increased severity and/or functional status of OA, whereas a decrease in OC resorption and/or an increase in apoptosis and/or OC expressed proteins as described herein could be indicative of amelioration of the disease state. So in one embodiment, the invention provides a method to monitor and determine the effectiveness of treatment regimes and/or to optimize treatment for a subject. In one embodiment the control can be values for OC apoptosis OC expressed proteins as described herein and resorption derived from a normal health subject or population (without OA) or potentially from a randomized sample of the population (with a known percentage of OA in said population), but in one embodiment from a health subject or healthy subject population (i.e., without OA). Wherein differences in values or degree of difference from normal or control values can be used to diagnose, predict OA disease state of a subject, including prognosis, functional status and/or severity of OA in said subject. The values for controls should be taken under the same or similar conditions as those taken for subject samples and/or standardized to enable comparatives. For instance if a PBMC sample is cultured under differentiating conditions to produce OC for 21 days in a subject sample, the control values should be obtained under similar conditions or the values between the subject sample and control values standardized, as required, to enable comparison for diagnostic and/or prognostic purposes.

The terms “sample”, “biological sample”, and the like mean a material known to or suspected of expressing or containing OC, OC precursors or markers of the presence of OC, differentiation capacity, activity or apoptosis of these cells. The test sample can be used directly as obtained from the source or following a pretreatment to modify the character of the sample. The sample can be derived from any biological source, such as tissues or extracts, including cells and physiological fluids, such as, for example, whole blood, plasma, serum, saliva, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins, DNA, and RNA may be isolated from the samples and utilized in the methods of the invention. In a preferred embodiment, the biological sample is serum or tissue extracts, most preferably serum or a fraction thereof, more peripheral blood mononuclear cells (PBMCs).

In one embodiment, the “subject” is a warm-blooded animal such as a mammal which is afflicted with or suspected to be afflicted with OA. Preferably, “subject” refers to a human.

In one embodiment the invention provides a method for diagnosing and/or predicting the degree of severity of OA in a subject comprising steps (a) to (c) above and then determining the various OC apoptosis, OC expressed protein levels and/or resorptive activity compared to a control, wherein an elevated resorptive activity or decrease in apoptosis or OC expressed protein levels correlates with OA and/or increased severity of OA.

In another embodiment, the invention provides a method for monitoring the progression of OA comprising conducting steps (a) to (c) above and comparing the values and/or results obtained with those previously obtained from the subject to monitor any changes in values of said indicators over time.

The methods and information derived from the methods of the invention can be used to determine and/or monitor a treatment regime for a patient and the effectiveness thereof. It can also be used to determine the prognosis, severity, functional status and presence of OA in a subject (for example the degree of variation of values from a control, the variation pattern of the OA markers over time or the values as compared to various values or ranges of known disease states). It can also be used to develop a database of values (mean, average, or range of values) of OC, apoptosis and resorption associated with a particular OA disease state. In one embodiment such a database could be used for diagnostic, prognostic and/or evaluative purposes as outlined herein. Further, in one embodiment the invention provides a use of the methods of the invention for treating OA by determining the OA disease state of a subject (such as, prognosis, severity and/or functional status) and then treating the subject in a manner appropriate for said disease state. In one embodiment, the progress and/or effectiveness of the treatment can be monitored using the methods of the present invention and the treatment adjusted as may be required to optimize treatment of a subject with OA.

The invention also relates to kits that can be used for carrying out all or part of the methods of the invention. In one embodiment, the kits can comprise one or more of the following: material for culturing osteoclast precursors from PBMCs (e.g.,: Ficoll-Hypaque gradient for PBMC isolation, tissue culture plates and cell culture media) and/or material for inducing OC differentiation (ie: RANKL, M-CSF) and/or material for evaluating the number of osteoclasts formed (reagents for Tartrate Resistant Acid Phosphatase staining), osteoclast apoptosis and bone resorption by osteoclasts and/or materials for detecting OC expressed protein markers of OA. In another embodiment the kit can comprise controls that can be used in the methods of the present invention. In one embodiment, the kit can comprise instructions for their use and/or for conducting the methods of the present invention, such as the diagnostic and/or prognostic methods of the invention as described herein, including but not limited to a method for determining the functional status and/or severity of OA in a subject.

The present invention will be further understood from the following non-limiting examples:

### EXAMPLES

### Example 1

### Osteoclast (OC) Generation

Fifty-two (52) subjects were enrolled in the study according to applicable laws. Patients satisfying the American College of Rheumatology (ACR) Criteria for knee OA (Altman R, Asch E, Bloch D, Bole G, Borenstein D, Brandt K, Christy W, Cooke T D, Greenwald R, Hochberg M, et al. Development of criteria for the classification and reporting of osteoarthritis. Classification of osteoarthritis of the knee. Diagnostic and Therapeutic Criteria Committee of the American Rheumatism Association. Arthritis Rheum. 1986 Aug. 29(8):1039-1049.) were recruited from the outpatient rheumatology clinic at the Centre Hospitalier Universitaire de Sherbrooke, in Sherbrooke Quebec. Control subjects were recruited from the local Sherbrooke, Quebec population. A summary of the demographics of the subjects enrolled in the studies described herein can be found at Table 1.

Blood samples were taken from the subjects. PBMCs were isolated from 50 ml of blood by Ficoll-Hypaque gradient (Boyum, Scand J Clin Lab Invest Suppl 1968; 97: 77-89) and the number of CD14+ osteoclast precursors was determined by FACS (Fluorescence activated cell sorter.

The whole population of PBMCs were cultured under differentiating conditions for 21 days in the presence of recombinant RANKL (75 ng/ml) and M-CSF (10 ng/ml) fixed, stained for TRAP (Tartrate Resistant Acid Phosphatase 5b) activity and for hematoxylin. The number of TRAP+ cells containing three or more nuclei-were counted in each well (Durand M, Gallant M A, de Brum-Fernandes A J., “Prostaglandin D2 receptors control osteoclastogenesis and the activity of human osteoclasts”, J Bone Miner Res. 2008 July; 23(7):1097-10) Triplicates were used for each patient.

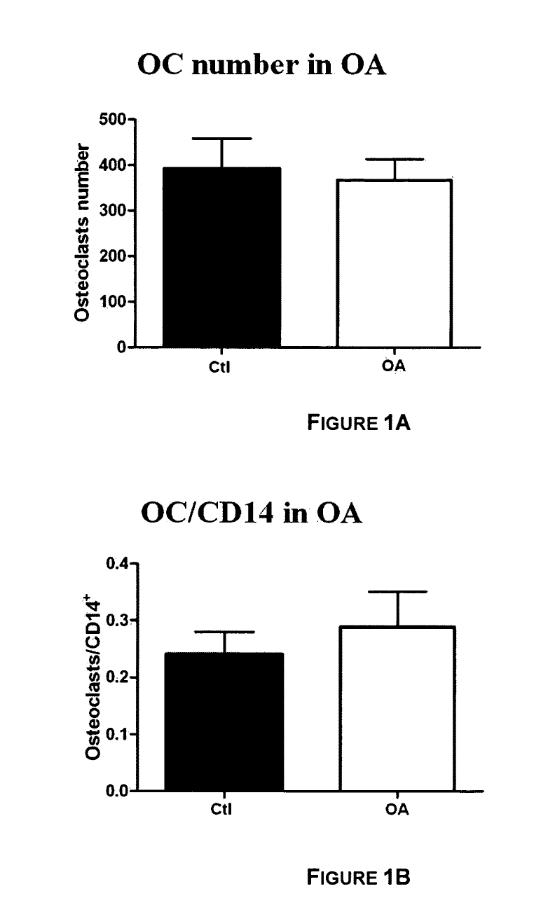

The studies demonstrate that the number of osteoclasts/well did not differ significantly between the control population (392.88±65.18) and OA (366.90±46.12) (FIG. 1A). FIG. 1B shows that the ratio OC generated/OC precursors (CD 14+ cells) is slightly but not significantly higher in subjects with OA (0.2887±0.0625) than in controls (0.2406±0.0389).

### Example 2

### OC Apoptosis

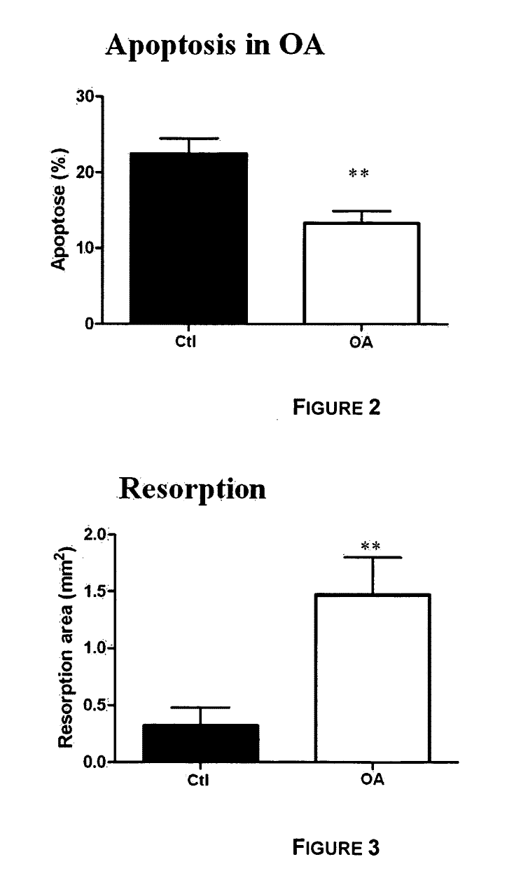

To study apoptosis, OCs were induced to differentiate from PBMC in culture for 21 days, as described above, then kept for 24 h without M-CSF or RANKL in 5% FBS. Cell death is visualized using the TACS Blue kit from R&D Systems and the percentage of OCs in apoptosis was recorded. FIG. 2 shows that, in the group of patients with OA, the percentage of OCs in apoptosis is significantly lower (13.26±1.65%) than that found in the control group (22.42±2.08%).

### Example 3

### OC Resorptive Activity

To directly assess OC resorptive activity, cells were differentiated for 21 days on bone slices, and stained for TRAP to assess OC number as described in Example 1 (above). After OC removal, bone slices were stained with 0.2% toluidine blue, and the total area of bone was quantified as described elsewhere (Durand M, Gallant M A, de Brum-Fernandes A J., “Prostaglandin D2 receptors control osteoclastogenesis and the activity of human osteoclasts”. J Bone Miner Res. 2008 July; 23(7):1097-10). FIG. 3 illustrates results of resorption activity studies, where higher resorption area reflects greater resorption OC activity. These results show that the group of patients with OA has higher resorption levels (9404.216±3556.814) than the control group (2030.456±961.157).

### Example 4

### Diagnosis of OA Severity

The correlation of OA disease severity to the number of osteoclasts generated in vitro and/or apoptosis and/or resportive activity of same can be studied. Disease severity can be defined by the Kellgren and Lawrence score method for knee OA. The primary outcome for knee OA is as defined by the American College of Rheumatology (Altman, R., E. Asch, D. Bloch, G. Bole, D. Borenstein, K. Brandt, W. Christy, T. D. Cooke, et al. 1986. Development of criteria for the classification and reporting of osteoarthritis. Classification of osteoarthritis of the knee. Diagnostic and Therapeutic Criteria Committee of the American Rheumatism Association. Arthritis Rheum 29:1039-1049.). The secondary outcome is disease severity. Because of the direct link between OCs and bone destruction one can chose radiographic parameters to define severe disease: The Kellgren and Lawrence method for knee OA is described in Kellgren, J. H. and J. S. Lawrence. 1957. Radiological assessment of osteo-arthrosis. Ann Rheum Dis 16:494-502. The radiographs can be examined and scored by a board certified rheumatologist blinded to the laboratory data. Since time of disease evolution will have an impact on the radiological scores, the number of years since the first symptoms will be used as a denominator. This gives a value that reflects the speed of progression of OA or severity. Patients above the average (or the median, depending on the distribution found for these variables) final score will be considered as having severe disease. Tertiary outcomes for OA can be determined using the functional status as defined by WOMAC (Bellamy, N., W. W. Buchanan, C. H. Goldsmith, J. Campbell and L. W. Stitt. 1988. Validation study of WOMAC: a health status instrument for measuring clinically important patient relevant outcomes to antirheumatic drug therapy in patients with osteoarthritis of the hip or knee. J Rheumatol 15:1833-1840). For OA populations osteoporosis will be defined according to DEXA and the WHO definition (Kanis, J. A., L. J. Melton, 3rd, C. Christiansen, C. C. Johnston and N. Khaltaev. 1994. The diagnosis of osteoporosis. J Bone Miner Res 9:1137-1141.). In phase 2 the primary outcome will be progression of the radiographic parameters; secondary_outcomes will be functional status for OA, as described above.

A cut-off can be used, where values equal to or above the cut-off indicate a subject with severe OA, while those below have less severe OA. To determine whether OC, OC apoptosis and/or OC resorption values in a patient correlate with severity of OA, PBMC samples are obtained from the subjects, differentiated for 21 days as described above under conditions that promote production of OCs and the mean number of OCs is determined for each group of subjects. Such a sample of OC PBMC derived cells can also be assayed for OC apoptosis and/or resorption activity. As such, obtaining a blood sample from a subject, culturing the PBMC cells to produce OCs and determine the number of OCs for the subject, OC apoptosis and/or OC resorption, as compared to a control or control values (e.g. for subjects without OA or values or value ranges established for patients with known OA severity) can be indicative of severity of OA.

### Example 5

### Use of OC Expressed Proteins as Indicators of OA

A summary of the demographics of the patients for the studies in Examples 5 and 6 can be found at Tables 2-4.

### Methods

A database of OA patients and controls were used. The patients in this study and that of Example 6 were recruited at the Division of Rheumatology, and all signed an informed consent. The control group was constituted of self-reported healthy individuals from the local (Sherbrooke) who volunteered to participate and signed informed consents. All clinical and laboratorial data are kept in a secure electronic database in Sherbrooke. To ensure that results would not be skewed due to age difference (i.e. to use data that were age matched), in one set of experiments the youngest controls were removed, in another the oldest OA patients were removed until the age did not differ statistically (at 5% significance) between the two populations. In the end 48 youngest controls, were removed or 30 oldest OA patients. In another set of data no controls, nor OA patients were removed. See Tables 2-4 for a summary of the demographics of the data sets.

More specifically, the original measurements were preprocessed to remove or correct noisy values, assure data type consistency and cross-link all records for the same patients. The entire population of 121 OA patients and 96 controls was processed to remove 45 patients (32 OA patients and 13 controls) that lack data for the RANK, IL-1R1, and IL-1R2 receptors and next the age of the two populations were matched in two ways:

1. The age-matched controls dataset was derived by removing 48 youngest controls until the age in the two populations is not statistically different when measured using group t-test at 5% significance. This resulted in the dataset with 89 OA patients and 35 controls.

2. The age-matched OAs dataset was derived by removing 30 oldest OA patients until the age in the two populations is not statistically different when measured using group t-test at 5% significance. This resulted in the dataset with 59 OA patients and 83 controls.

For each of the three datasets (the full dataset with no patients or controls removed, the age-matched controls dataset, and the age-matched OAs dataset) statistical tests were performed for each of potential markets using 2 steps:

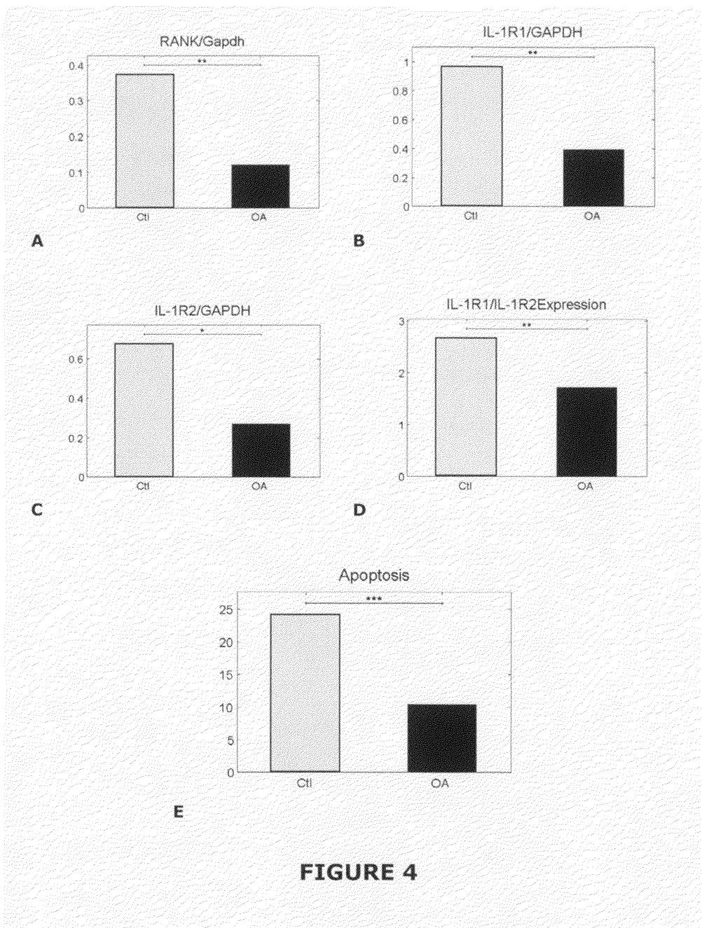

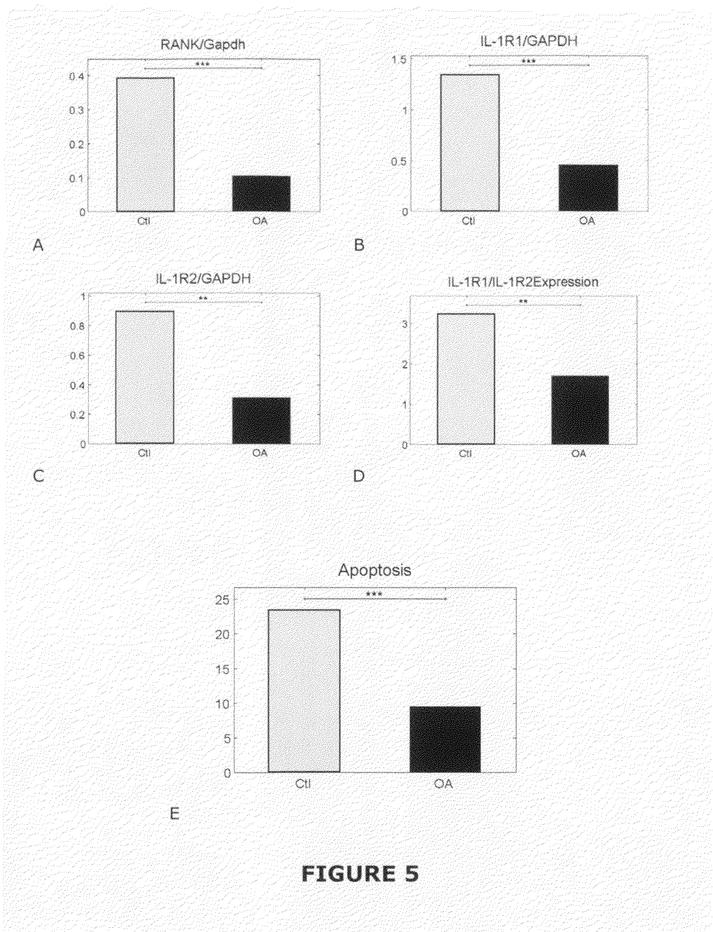

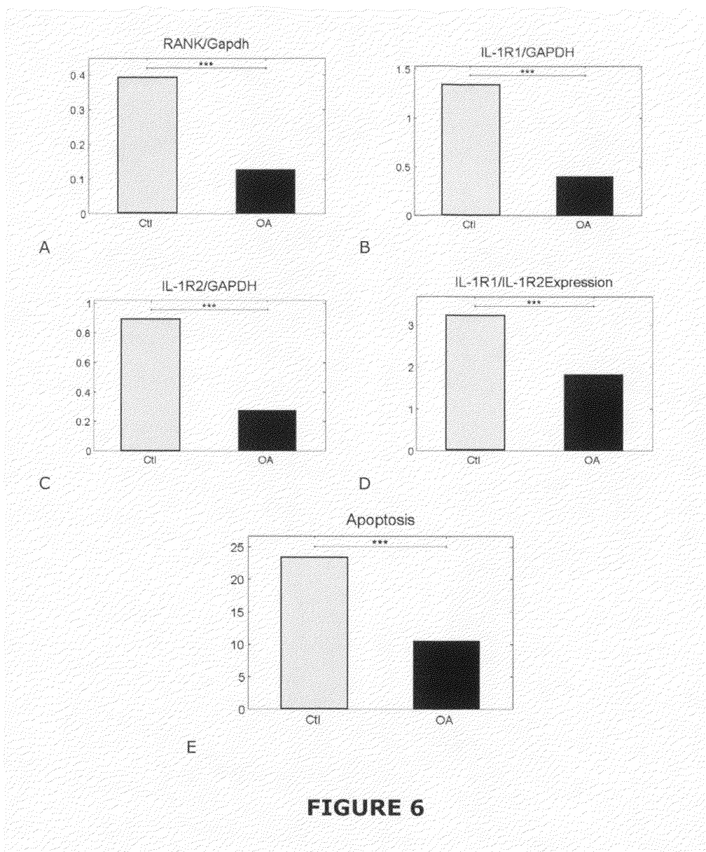

1. Distribution of values of a given marker was tested using Shapiro-Wilk test (Shapiro, S. S. and Wilk, M. B. (1965). “An analysis of variance test for normality (complete samples)”, Biometrika, Vol. 52, No. 3/4, pages 591-611.)
2. For a normal attribute (according to the Shapiro-Wilk test) we tested the significance of the differences using group t-test; for a non-parametric attribute (according to the Shapiro-Wilk test) we used the Mann-Whitney test (Mann, H. B., & Whitney, D. R. (1947). “On a test of whether one of two random variables is stochastically larger than the other”. Annals of Mathematical Statistics, 18, 50-60.)
The results are presented using bar charts where vertical bars denote the average value for a given patient group, groups are annotated on the x-axis and color-coded, and horizontal bars at the top of each diagram show statistical difference at a given P value (*** denotes P<0.001; ** denotes P<0.01; and * denotes P<0.05) between the two spanned groups. FIG. 4 represents the graph wherein young control subjects were removed (for the age-matched controls dataset), FIG. 5 represents the graph where oldest OA patients were removed (for the age-matched OAs dataset), and FIG. 6 represents the graph for the full dataset with no patients or controls removed.

### Apoptose

The procedure as used in Example 2 and as further described below was used.

### Protein Isolation

Monocytes were plated in the presence of M-CSF and RANKL, after 21 days, protein was isolated from OCs using RIPA buffer [50 mM Tris, 150 mM NaCl, 1% Triton-X, 1% SDS, 0.5% sodium deoxycholate]. Briefly, cells were washed with cold PBS-Mg²⁺and -Ca²⁺. Cells were then scraped into approximately 1 mL RIPA+ complete protease inhibitor cocktail (Roche) which were added to cell culture dishes. The whole cell lysate was left on ice 20 minutes with vortexing, then centrifuged at ˜14 000 rpm at 4° C., for 15 minutes. Protein concentrations were determined and then used to adjust the amounts for immunoblotting.

Cell lysates (50 μg or 50 μL, whichever was available) were run on 10% mini SDS-PAGE gels. Protein was transferred to nitrocellulose (Amersham Hybond-ECL) using full immersion transfer apparatus (Bio-Rad) on ice at 75 V, 150 mAmps for 1 hour (or 130 mAmps for 1.5 hours). Transfer was confirmed by staining with 0.2% Ponceau-S in acetic acid. Blots were then blocked in Tris buffered saline Tween-20 (TBS-T)+5% milk solution for 1 hour. Primary antibodies were added at appropriate dilutions in TBS-T+5% milk and the blots incubated at 4° C. 0/N. Receptors were detected with antibodies against RANK (R&D Systems), IL-1R1 (Santa Cruz) and IL-1R2 (R&D Systems). GAPDH was detected by probing with anti-GAPDH (Abeam) and was used as a control. Blots were then washed in TBS-T, after which appropriate secondary antibody was added at concentrations of 1:1000-1:5000 in TBS-T+ milk incubated at room temperature for 1 hour. Blots were then washed and developed using ECL reagents (Perkin Elmer). Images were captured using GeneSnap (Syngene) or ChemiDoc XRS HQ2 (Biorad). Quantification and analysis were performed using GeneTools (Syngene) as previously described (Manolson M F, Yu H, Chen W, Yao Y, Li K, Lees R L, Heersche J N 2003 The a3 isoform of the 100-kDa V-ATPase subunit is highly but differentially expressed in large (≧10 nuclei) and small (≦5 nuclei) osteoclasts. J Biol Chem 278(49):49271-8.) or Quantity One® using the rolling disk band analysis application (Biorad). Briefly, to ensure that the chemiluminescent signals from the immunoblots were within the linear range, each protein sample. Multiple exposure times were recorded using a CCD camera, and an exposure was used for quantification only if the obtained signal reflected the serial dilution of the sample. For each separate gel, the absolute value obtained at each of the four protein concentrations was divided by the absolute value obtained for GAPDH. GAPDH was used to normalize the signal between different samples as in situ hybridization had revealed no difference in GAPDH gene expression between large and small OCs (Manolson M F, Yu H, Chen W, Yao Y, Li K, Lees R L, Heersche J N 2003 The a3 isoform of the 100-kDa V-ATPase subunit is highly but differentially expressed in large (≧10 nuclei) and small (≦5 nuclei) osteoclasts. J Biol Chem 278(49):49271-8).

### Results

FIG. 4 illustrates data for the dataset with the age-matched controls (35 controls and 89 OA patients): OA patients express lower levels of RANK, IL-1R1, and IL-1R2 receptors, and decreased apoptosis compared to controls. Human blood monocytes drawn from blood of OA or control patients were differentiated into OCs in the presence of M-CSF and RANKL. Cell lysates from individual patient OCs were then collected and run on 10% SDS-PAGE gels, and immunoblotted for RANK receptor, IL-1R1, IL-1R2 and GAPDH. Protein expression from immunoblots was quantified using a CCD camera (Syngene or BioRad) and densitometry software. Expression levels were normalized to levels of GAPDH to account for inconsistencies in loading. Numbers reflect the mean ratio of expression of RANK/GAPDH (A), IL-1R1/GAPDH (B), or IL-1R2/GAPDH (C) from all patients in OA groups and control groups. Expression levels of IL-1R1 and IL-1R2 levels were compared by ratioing IL-1R1 and IL-1R2 levels and comparing means of combined control and OA patients (D). Plated OCs were measured for apoptosis using a TUNEL based assay (TACS Blue Kit from R&D systems). Data is expressed as the percentage of apoptotic OCs (E). *p<0.05; **p<0.01; ***p<0.001.

FIG. 5 illustrates data with for the dataset with the age-matched OA patients (83 controls and 59 OA patients): OA patients express lower levels of RANK, IL-1R1, and IL-1R2 receptors, and decreased apoptosis compared to controls. Human blood monocytes drawn from blood of OA or control patients were differentiated into OCs in the presence of M-CSF and RANKL. Cell lysates from individual patient OCs were then collected and run on 10% SDS-PAGE gels, and immunoblotted for RANK receptor, IL-1R1, IL-1R2 and GAPDH. Protein expression from immunoblots was quantified using a CCD camera (Syngene or BioRad) and densitometry software. Expression levels were normalized to levels of GAPDH to account for inconsistencies in loading. Numbers reflect the mean ratio of expression of RANK/GAPDH (A), IL-1R1/GAPDH (B), or IL-1R2/GAPDH (C) from all patients in OA groups and control groups. Expression levels of IL-1R1 and IL-1R2 levels were compared by ratioing IL-1R1 and IL-1R2 levels and comparing means of combined control and OA patients (D). Plated OCs were measured for apoptosis using a TUNEL based assay (TACS Blue Kit from R&D systems). Data is expressed as the percentage of apoptotic OCs (E). *p<0.05; **p<0.01; ***p<0.001.

FIG. 6 illustrates data for all control and OA patients (no patients or controls removed): OA patients express lower levels of RANK, IL-1R1, and IL-1R2 receptors, and decreased apoptosis compared to controls. Human blood monocytes drawn from blood of OA or control patients were differentiated into OCs in the presence of M-CSF and RANKL. Cell lysates from individual patient OCs were then collected and run on 10% SDS-PAGE gels, and immunoblotted for RANK receptor, IL-1R1, IL-1R2 and GAPDH. Protein expression from immunoblots was quantified using a CCD camera (Syngene or BioRad) and densitometry software. Expression levels were normalized to levels of GAPDH to account for inconsistencies in loading. Numbers reflect the mean ratio of expression of RANK/GAPDH (A), IL-1R1/GAPDH (B), or IL-1R2/GAPDH (C) from all patients in OA groups and control groups. Expression levels of IL-1R1 and IL-1R2 levels were compared by ratioing IL-1R1 and IL-1R2 levels and comparing means of combined control and OA patients (D). Plated OCs were measured for apoptosis using a TUNEL based assay (TACS Blue Kit from R&D systems). Data is expressed as the percentage of apoptotic OCs (E). *p<0.05; **p<0.01; ***p<0.001.

It can be seen the results of the age matched control (FIGS. 4 and 5) were similar to those of that was not age matched (e.g. FIG. 6)

### Example 6

### Use of Multiple Markers of OA in a Diagnostic Test

Example 6 shows how each of the proteins could be used as indicators of OA, In this example, it is shown that using the results of more than one OC expressed protein can improve accuracy of OA diagnosis.

The source of the data and methodology was the same as that of Example 5.

Example 5 shows that each of the surrogate markers, including apoptosis, IL-1R1, IL-1R2 and RANK, could be used as indicators/markers of OA. In this example, it is shown that using the results of more than one marker can improve accuracy of OA diagnosis. The apoptosis marker was combined with the other three surrogate markers and the combination of the apoptosis with RANK and the combination of apoptosis with IL-1R1/IL-1R2 ratio exhibit favorable diagnostic accuracy when compared with other combinations of two markers. It was also demonstrated that usage of two markers in tandem results in a better diagnostic tool (when measured using accuracy) than when only one marker, i.e., apoptosis that is characterized by the strongest and most consistent (between the three datasets) statistical difference between OA patients and normals among the surrogate markers, is used.

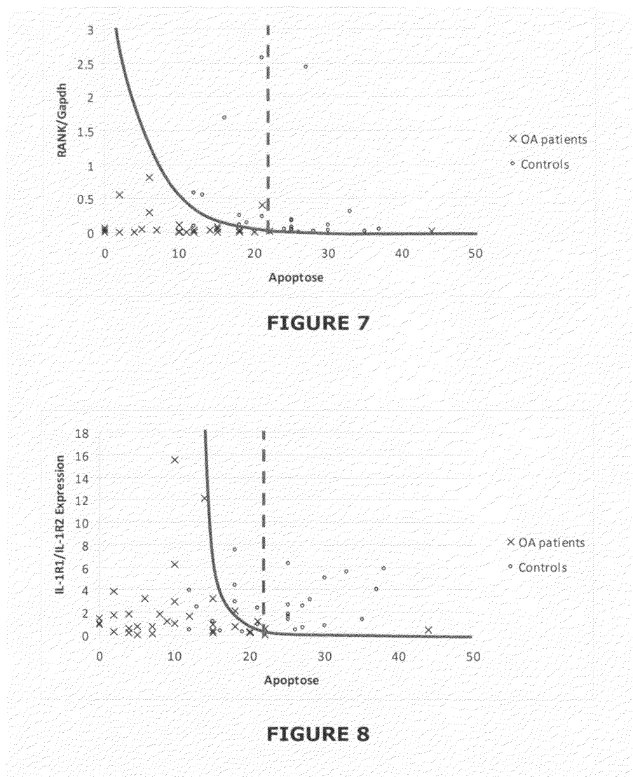

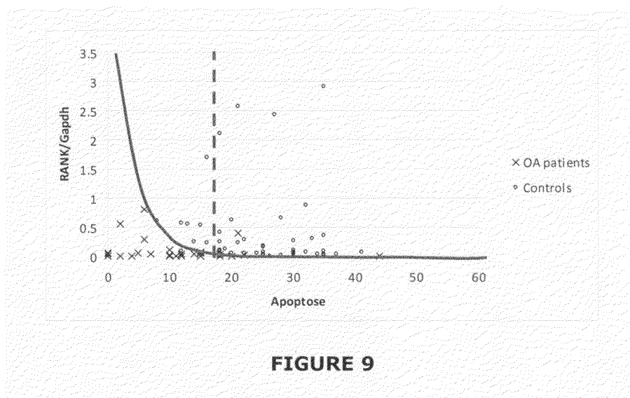

FIG. 7 represents the graph wherein the youngest control subjects were removed, while FIG. 8 represents the graph where oldest OA patients were removed. FIG. 9 represents the graph with no controls nor OA patients removed.

FIG. 7 illustrates results using data wherein the youngest control subjects were removed—the age-matched controls and OA patients (35 controls and 89 OA patients) showing the apoptosis on x-axis and RANK on y-axis. The OA patients are denoted using “x” markers and the controls are shown using “o” markers. The plot shows that patients with Apoptose <11.5 are likely to have OA (only OA patients have apoptose <11.5), and that patients with apoptose>=23 are likely to be controls (only controls have apoptose>=11.5). The subjects with the apoptose between 11.5 and 23 include both controls and OA patients. A diagnostic tool based on apoptosis in which it is assumed that apoptose<=22 (which is the optimal threshold for the age-matched controls dataset) indicates OA patients and otherwise it would be assumed that a given subject is a control, would provide about 85% accuracy (success rate) on the age-matched controls dataset. This diagnostic tool is demonstrated using a vertical dashed line, i.e., points to the left of the line are assumed to be OA patients and points to the right of the line are assumed to be controls. Inclusion of RANK improves the accuracy of the diagnostic tool. The figure shows that for the apoptose between 11.5 and 23, higher values of RANK are indicative of controls while lower values of RANK are indicative of OA patients. A diagnostic tool based on both apoptosis and RANK represented by the solid line, in which it is assumed that points above the line are controls and points below the line are OA patients, provides about 90% accuracy (success rate) on the age-matched controls dataset. This demonstrates that combination of apoptosis and RANK provides better predictive quality.

FIG. 8 illustrates results using data that was age-matched where the oldest OA patients were removed: controls and OA patients (35 controls and 89 OA patients), showing the apoptosis on x-axis and IL-1R1/IL-1R2 expression on y-axis. The OA patients are denoted using “x” markers and the controls are shown using “o” markers. A diagnostic tool based on apoptosis in which it is assumed that apoptose<=22 (which is the optimal threshold for the age-matched controls dataset) would indicate OA patients and otherwise it would be assumed that a given subject is a control, would provide about 85% accuracy (success rate) on the age-matched controls dataset. This diagnostic tool is demonstrated using a vertical dashed line, i.e., points to the left of the line are assumed to be OA patients and points to the right of the line are assumed to be controls. Inclusion of IL-1R1/IL-1R2 expression improves the accuracy of the diagnostic tool. A diagnostic tool based on both apoptosis and IL-1R1/IL-1R2 expression represented by the solid line, in which it is assumed that points above the line are controls and points below the line are OA patients, would provide about 89% accuracy (success rate) on the age-matched controls dataset. This demonstrates that combination of apoptosis and IL-1R1/IL-1R2 expression provides better predictive quality.

FIG. 9 illustrates results using the full dataset with no patients or controls removed (96 controls and 121 OA patients) showing the apoptosis on x-axis and RANK on y-axis. The OA patients are denoted using “x” markers and the controls are shown using “o” markers. A diagnostic tool based on apoptosis in which it is assumed that apoptose <=17 (which is the optimal threshold for the full dataset) would indicate OA patients and otherwise it would assume that a given subject is a control, would provide about 77% accuracy (success rate) on the full dataset. This diagnostic tool is demonstrated using a vertical dashed line, i.e., points to the left of the line are assumed to be OA patients and points to the right of the line are assumed to be controls. Inclusion of RANK improves the accuracy of the diagnostic tool. A diagnostic tool based on both apoptosis and RANK represented by the solid line, in which it is assumed that points above the line are controls and points below the line are OA patients, provides about 86% accuracy (success rate) on the full dataset. This demonstrates that combination of apoptosis and RANK provides better predictive quality.

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

TABLE 1
Baseline characteristics of the controls and patients.†

| | Control (Ctl) | OA |
|---|---|---|
| Characteristics | (N = 41) | (N = 52) |
| Age -- yr | 57.1 ± 1.2 | 67.8 ± 1.4*** |
| Female sex -- no. (%) | 23 (56.1) | 38 (73.1) |
| Menopause -- no. (%) | | |
| Menopause | NA | 26 (68.4) |
| Pre-menopause | NA | 1 |
| Body-mass index‡ | 27.6 ± 0.9 | 30.8 ± 1.0* |
| Ethnic group -- no. (%)¶ | | |
| Caucassian | 38 (92.7) | 52 (100) |
| Other | 3 | 0 |
| Smoking status -- no. (%) | | |
| Ever smoke | 15 (36.6) | 27 (51.9) |
| Alcohol status -- no. (%) | 32 (78.0) | 28 (53.8)* |

†Plus-minus values are means ± SEM.
‡Body-mass index is the weight in kilograms divided by the square of the height in meters.
¶Ethnic group are self-reported.
*P ≦ 0.05 for the comparison between this group and the control group.
***P ≦ 0.001 for the comparison between this group and the control group.

TABLE 2
Data Set For Experiments Where Youngest Controls Were Removed

| Characteristics | Controls (N = 35) | OA (N = 89) |
|---|---|---|
| Age - years (standard deviation.) | 65.6 (3.8) | 67.9 (9.2) |
| Sex | Female 80 (65%), Male 44 (35%) | |
| Ethnic group | Caucasians 121 (98%), Other 2 (2%) | |
| Weight - kg (standard deviation) | 75.4 (16.2) | 80.9 (19.4) |
| Height - cm (standard deviation) | 166.1 (9.4) | 164.1 (8.4) |
| Smoking status | | |
| No. years of smoking - years | 12.8 (17.7) | 12.8 (15.3) |
| (standard deviation) | | |
| No. years non-smoking - years | 7.9 (12.8) | 10.0 (13.0) |
| (standard deviation) | | |

Baseline characteristics for controls and OA patients for the dataset with the age-matched controls (age was matched by removing youngest controls until the differences in age between the control and OA population is not statistically significant when measured using group t-test at 5% significance) with 35 controls and 89 OA patients.
The age, weight, height, and smoking are given as the average in each population with the corresponding standard deviations.
“No. years of smoking” refers to the duration of present or past smoking history, and
“No. years non-smoking” refers to the number of years since cessation of smoking.
We also show the distribution of sex and ethnic groups.
The ethnic groups are self-reported and this information is not available for 1 patient.

TABLE 3
Data Set For Experiments Where Oldest OA Patients Were Removed

| Characteristics | Controls (N = 83) | OA (N = 59) |
|---|---|---|
| Age - years (standard deviation.) | 60.9 (5.3) | 63.0 (7.0) |
| Sex | Female 90 (63%), Male 52 (37%) | |
| Ethnic group | Caucasians 138 (97%), Other 2 (1%) | |
| Weight - kg (standard deviation) | 74.3 (13.9) | 83.0 (20.2) |
| Height - cm (standard deviation) | 165.7 (8.7) | 163.8 (8.1) |
| Smoking status | | |
| No. years of smoking - years | 9.9 (15.7) | 12.6 (14.3) |
| (standard deviation) | | |
| No. years non-smoking - years | 4.7 (10.2) | 8.3 (10.5) |
| (standard deviation) | | |

Baseline characteristics for controls and OA patients for the dataset with the age-matched OA patients (age was matched by removing oldest OA patients until the differences in age between the control and OA population is not statistically significant when measured using group t-test at 5% significance) with 83 controls and 59 OA patients.
The age, weight, height, and smoking are given as the average in each population with the corresponding standard deviations.
“No. years of smoking” refers to the duration of present or past smoking history, and
“No. years non-smoking” refers to the number of years since cessation of smoking.
We also show the distribution of sex and ethnic groups.
The ethnic groups are self-reported and this information is not available for 2 patients.

TABLE 4
Data Set For Experiments Where No Controls and No OA Patients
Were Removed.

| Characteristics | Controls (N = 96) | OA (N = 121) |
|---|---|---|
| Age - years (standard deviation) | 60.8 (5.7) | 67.2 (9.0) |
| Sex | Female 135 (62%), Male 82 (38%) | |
| Ethnic group | Caucasians 212 (98%), Other 3 (1%) | |
| Weight - kg (standard deviation) | 72.9 (14.3) | 81.5 (18.6) |
| Height - cm (standard deviation) | 165.8 (8.6) | 164.9 (8.1) |
| Smoking status | | |
| No. years of smoking - years | 9.9 (15.4) | 12.8 (15.5) |
| (standard deviation) | | |
| No. years non-smoking - years | 5.0 (10.5) | 9.7 (12.7) |
| (standard deviation) | | |

Baseline characteristics for controls and OA patients for the full dataset (no patients or controls removed) with 96 controls and 121 OA patients.
The age, weight, height, and smoking are given as the average in each population with the corresponding standard deviations.
“No. years of smoking” refers to the duration of present or past smoking history, and
“No. years non-smoking” refers to the number of years since cessation of smoking.
We also show the distribution of sex and ethnic groups.
The ethnic groups are self-reported and this information is not available for 2 patients.

What is claimed is:

1. A method for diagnosing, predicting severity of, and monitoring osteoarthritis in a subject comprising obtaining osteoclasts derived from peripheral blood mononuclear cells from a sample from the subject and assaying them for OA markers selected from the group consisting of osteoclast apoptosis and/or osteoclast expressed proteins and/or osteoclast resorptive activity wherein a decrease in osteoclast apoptosis and/or osteoclast expressed proteins and/or an increase in osteoclast resorptive activity compared to a control sample is indicative of osteoarthritis.

2. A method as claimed in claim 1 wherein osteoclast apoptosis is assayed and a decrease in osteoclast apoptosis compared to a control sample is indicative of osteoarthritis.

3. A method as claimed in claim 1 wherein osteoclast resorptive activity is assayed and an increase in osteoclast resorptive activity compared to a control sample is indicative of osteoarthritis.

4. A method as claimed in claim 1 wherein an increase in osteoclast resorption or a decrease in OC apoptosis reflects disease severity and/or progression rates of joint destruction.

5. A method of claim 1 when the osteoclast expressed proteins are selected from one or more of the following: IL-1R1, IL-1R2; IL-1R1/IL-1R2; and RANK.

6. A method of claim 1 where the values of said proteins are expressed in terms of a ratio with a control.

7. The method of claim 6 wherein the control is GADPH.

8. The method of claim 1 where two of the markers are used.

9. The method of claim 8 wherein the two markers are apoptose and an OC expressed protein.

10. The method of claim 9 wherein the OC expressed protein is selected from the group consisting of: IL-1R1, IL-1R2; IL-1R1/IL-1R2; and RANK.

11. The method of claim 10 wherein the two markers are selected from the group consisting of apoptose, RANK and IL-1R1/IL-1R2.

12. The method of claim 11 wherein the two markers are apoptose and RANK.

13. The method of claim 11 wherein the two markers are apoptose and IL-1R1/IL-1R2.

14. A method of claim 10 where the values of said proteins are expressed in terms of a ratio with a control.

15. The method of claim 14 wherein the control is GADPH.
